(19) United States
(12) Patent Application Publication
(10) Pub. No.: US 2015/0274655 A1
(43) Pub. Date: Oct. 1, 2015

(54) POLYSULFIDE MIXTURE, METHOD FOR PRODUCING SAME, AND USE OF THE POLYSULFIDE MIXTURE IN RUBBER MIXTURES

(71) Applicant: LANXESS Deutschland GmbH, Cologne (DE)

(72) Inventors: Ulrich Feldhues, Bergisch Gladbach (DE); Heinz Unterberg, Dormagen (DE); Hermann-Josef Weidenhaupt, Pulheim (DE); Melanie Wiedemeier-Jarad, Dormagen (DE)

(21) Appl. No.: 14/434,899

(22) PCT Filed: Oct. 29, 2013
(86) PCT No.: PCT/EP2013/072599

§ 371 Date: Apr. 10, 2015

(30) Foreign Application Priority Data
Oct. 31, 2012 (EP) 12190588.9

(51) Int. Cl. C07C 323/62; C08K 5/36; C08K 5/548; C07C 319/24; C08L 9/06

(52) U.S. Cl. CPC C07C 323/62; C07C 319/24; C08L 9/06; C08K 5/548; C08K 5/36; C08L 2205/02; C08L 2205/03; C08L 2205/06

(57) ABSTRACT

The present invention relates to polysulfide mixtures comprising two or more compounds of the formula (I),

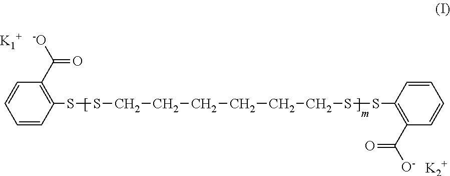

where the cations K₁⁺ and K₂⁺ mutually independently are any desired monovalent or are the nth part of an n-valent cation, and m is 0, 1, and/or 2, to a process for the production of these polysulfide mixtures, to the use of the polysulfide mixtures in rubber mixtures, to the rubber mixtures, to rubber vulcanizates produced therefrom, and to the use of these.

The present invention relates to novel polysulfide mixtures, to a process for the production of these polysulfide mixtures, to the use of the polysulfide mixtures in rubber mixtures, to rubber vulcanizates produced therefrom, and to use of these.

Silica-containing rubber mixtures are important starting materials, for example for the production of tires with reduced rolling resistance. These require less rolling deformation energy (than tires which comprise only carbon black as filler), and therefore reduce fuel consumption. Because various states have decided on compulsory marking to indicate rolling resistance on tires, there is a high level of interest in achieving a further reduction in this resistance.

As mentioned by way of example in DE 2 255 577 A, polysulfidic silanes are used as reinforcing additives to improve the physical properties of vulcanizates. The property profile of the resultant rubber vulcanizates is not yet ideal. A particularly desirable feature, alongside improved rolling resistance, is low viscosity (Mooney viscosity ML 1+4/100° C.) of the rubber mixture; this improves processability. Other additional substances have been proposed for this purpose, examples being fatty acid esters, fatty acid salts, and mineral oils; although these increase flowability they simultaneously reduce the moduli of the vulcanizates at relatively high elongation (e.g. from 100% to 300%), or else their hardness, and some of the reinforcing effect of the filler is then therefore lost. However, inadequate hardness or stiffness of the vulcanizate leads to unsatisfactory running performance of the tire, particularly around curves. The hardness of the vulcanizate can be increased by increasing the proportion of reinforcing filler, or by reducing the proportion of plasticizer oil, but each of these two measures has the disadvantage of higher mixture viscosity during processing.

EP 0 489 313 describes additives comprising glycol functions and having good mechanical properties and improved hysteresis performance. However, in comparison with bis[3-(triethoxysilyl)propyl]tetrasulfide according to DE-OS (German Published Specification) 2 255 577 the examples reveal no improvement of rolling resistance (tan δ at 60° C.).

EP 1 000 968 achieved an Improvement in physical properties via use of a polysulfidic silane in combination with specific antireversion agent in SBR, but no significant change in comparison with the prior art was achieved here either in the viscosity of the mixture or in rolling resistance (tan δ at 60° C.).

The application EP 11164319, hitherto unpublished, describes the use of a compound with the Idealized structure (II). With reference to the formulation in EP 11164319 it is obvious to the person skilled in the art in the sector of polysulfide compounds—in view of the difficulty of separating polysulfides—that a mixture of various compounds is involved, corresponding approximately to the formula (II) when an average is taken across the number of sulfur atoms and alkylene bridges. No analysis of the composition of the mixture was carried out.

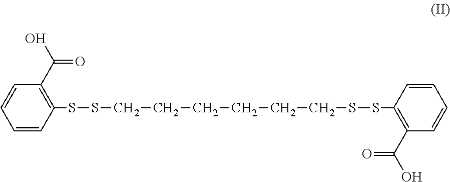 [SMILES: O=C(O)c1ccccc1SSCCCCCCSSc1ccccc1C(=O)O]

Abrasion and rolling resistance were poorer than those of a reference mixture without the compound with the idealized structure (II). There was moreover an undesirable increase in the Mooney scorch time at 130° C.

It is an object of the present invention to provide novel rubber additives, a process for production of these, and novel rubber mixtures which, with identical or improved flowability of the rubber mixtures, can be converted to vulcanizates with reduced rolling resistance, but where, at the same time, the similarly important parameters Shore A hardness, 300 modulus, elongation at break, tensile strength, and abrasion exhibit no substantial impairment.

Subsequent work on the mixture disclosed in EP 11164319 revealed that this comprises, alongside 54% of the compound of the formula (II), 25% of 2,2′-dithiobisbenzoic acid and 21% of a compound in which a S₂—(CH₂)₆—S₂—(CH₂)₆—S₂ chain connects the two benzoic acid moieties respectively in 2-position.

Surprisingly, it has now been found that a narrower distribution of the S—(CH₂)₆—S units in the polysulfide mixture markedly improves the property profile of rubber mixtures comprising this polysulfide mixture, and of the vulcanizate thereof, and thus achieves the object of the Invention.

It has moreover been found that the desired properties of the rubber mixtures and of vulcanizates obtained therefrom are achieved not only by using the compounds with the Idealized structure (II) but also by using salts of these compounds, in particular zinc salts.

The present invention therefore provides polysulfide mixtures comprising two or more compounds of the formula (I),

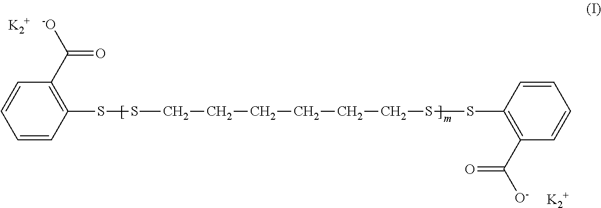

where the cations K₁⁺ and K₂⁺ mutually independently are any desired monovalent or the nth part of any desired n-valent cation, and m is 0, 1 and/or 2, characterized in that the total proportion of compound(s) where m=1 is at least 80%, preferably at least 85%, particularly preferably at least 90%, very particularly preferably from 93 to 99%, based on the total quantity of polysulfide compounds of the formula (I). For the purposes of the present application, the quantitative data relating to the compounds of the formula (I) are area percentage data from the type of HPLC measurement described near the end of example 3, using UV detector.

It is preferable that the cations K₁⁺ and K₂⁺ are mutually independently H⁺, are an alkali metal cation, in particular Li⁺, Na⁺, K⁺, ½ alkaline earth metal cation, in particular ½Mg²⁺, Ca²⁺, ⅓Al³⁺, or are the nth part of an n-valent rare earth metal cation, or are ½Zn²⁺.

It is very particularly preferable that the cations K₁⁺ and K₂⁺ mutually independently are H⁺ or are ½Zn²⁺, in particular being ½Zn²⁺.

Polysulfide mixtures of the invention typically comprise a total of up to 10%, preferably up to 7.5%, particularly preferably up to 5%, and very particularly preferably no more than 3%, of polysulfide compounds of the formula (I) where m=0, based on the total quantity of polysulfide compounds of the formula (I).

Polysulfide mixtures of the invention correspondingly usually comprise a total of up to 10%, preferably up to 7.5%, particularly preferably up to 5%, and very particularly preferably less than 2%, of polysulfide compounds of the formula (I) where m=2, based on the total quantity of polysulfide compounds of the formula (I).

The total proportion of polysulfides of the formula (I) where m=0 and/or m=2 in the polysulfide mixtures of the invention is up to 20%, preferably up to 15%, particularly preferably up to 10%, and very particularly preferably from 1 to 7%, based on the total quantity of polysulfide compounds of the formula (I).

Elemental analysis generally gives sulfur content of from 22 to 32% for polysulfide mixtures of the invention, preferably from 24 to 30%, and particularly preferably from 27 to 29%.

The average number of sulfur atoms in the molecules of the formula (I) in the polysulfide mixtures according to the invention is normally from 3.5 to 4.5, preferably from 3.7 to 4.3, particularly preferably from 3.8 to 4.2, and very particularly preferably from 3.9 to 4.1.

In one preferred embodiment, the polysulfide mixture of the invention comprises less than 10%, particularly less than 3%, very particularly less than 1%, of byproducts or admixtures, i.e. compounds not corresponding to the formula (I).

The quantity of admixed elemental sulfur in the polysulfide mixture of the invention is typically less than 2%, preferably less than 1%, particularly preferably less than 0.3%, very particularly preferably less than 0.1%, and most preferably zero.

The quantity of admixed accelerators of the mercapto group or of the sulfenamide group in the polysulfide mixture of the Invention is typically less than 2%, preferably less than 1%, particularly preferably less than 0.3%, very particularly preferably less than 0.1%, and most preferably zero.

The total chlorine content of a polysulfide mixture of the invention is typically <1%, preferably <1000 ppm, particularly preferably <200 ppm, very particularly preferably <50 ppm, most preferably <10 ppm.

The present invention also comprises a process for the production of the polysulfide mixtures of the invention by bringing 2-mercaptobenzoic acid or its salts (III) into contact with hexamethylene 1,6-bisthiosulfates (IV).

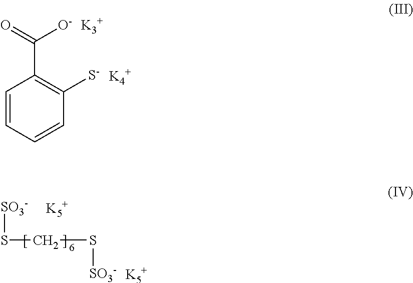

The cations K₃⁺ and K₄⁺ here mutually independently are any desired monovalent cations or are the nth part of any desired n-valent cation, preferably being H, an alkali metal cation, in particular Li⁺, Na⁺, K⁺, ½ alkaline earth metal cation, in particular ½Mg²⁺, ½Ca²⁺, ⅓Al³⁺, or are the nth part of an n-valent rare earth metal cation, or are ½Zn²⁺, particularly preferably being H⁺, Na⁺, K⁺ or being ½Zn²⁺. The two cations K₅⁺ here are identical or different, preferably identical, and mutually independently are any desired manovalent cations, or are the nth part of any desired n-valent cation, preferably being an alkali metal cation, in particular Li⁺, Na⁺, K⁺, ½ alkaline earth metal cation, in particular ½Mg²⁺, ½Ca²⁺, ⅓Al³⁺ or are the nth part of an n-valent rare earth metal cation, or are ½Zn²⁺, particularly preferably being Na⁺.

In one particularly preferred embodiment, disodium hexamethylene 1,6-bisthiosulfate dihydrate, which is obtainable commercially, is used as compound of the formula (IV).

Compound(s) of the formula (III) is/are usually brought into contact with compound(s) of the formula (IV) in an aqueous or aqueous-organic medium, and an Inert gas atmosphere is advantageous here in order to avoid oxidation products.

The aqueous-organic medium here comprises water and one or more organic solvents, in particular solvents of the group of alcohols, esters and ethers.

Inert gases used can be any gases which exhibit no, or only insubstantial, reaction under reaction conditions. It is preferable to use noble gases or nitrogen.

In one preferred embodiment, the contact is achieved in the presence of aldehydes and/or ketones, in particular in the presence of formaldehyde.

It is preferable that the contact is effected at temperatures in the range from −5° C. to 19° C., preferably in the range from 0° C. to 15° C., particularly preferably in the range from 0° C. to 10° C. Excessively high temperatures and long reaction times, and long continued-stirring times, lead to increasing quantities of byproducts. Compounds of the formula (I) where m=1 can be produced with very high selectivity at temperatures in the range from 0 to 10° C. At temperatures a ≧20° C., the selectivity for this substance is already reduced, and in particular in the case of relatively long reaction times the proportion of products of the formula (I) where m=1 decreases, while the proportion of compounds of the formula (I) where m=0 increases, but when these are used in rubber the desired reduction of rolling resistance of the vulcanizate is not achieved.

In one preferred embodiment, the aqueous medium, compound(s) (IV), and formaldehyde are used as initial charge, and 2-mercaptobenzoic acid (III) in the form of aqueous solution of its salts, i.e. compound(s) of the formula (III), where K₃⁺ and/or K₄⁺ are not H⁺, is/are metered into the mixture, where the pH is kept in the range from 7 to 13, particularly preferably from 8 to 12, in particular from 9 to 11, preferably via addition of a Bronsted acid. This can be added in any desired concentration, but preferably in dilute form. Particular preference is given to use of one or more mineral acids. It is preferable that the pH is adjusted via subsequent addition of further acid to the range from 0 to 4, in particular from 1 to 3. It is thus possible to precipitate the compounds of the formula (V) in which m is 0, 1, and/or 2, and to obtain polysulfide mixtures of the invention based on polysulfides of the formula (I), where K₁⁺ and K₂⁺ are H⁺.

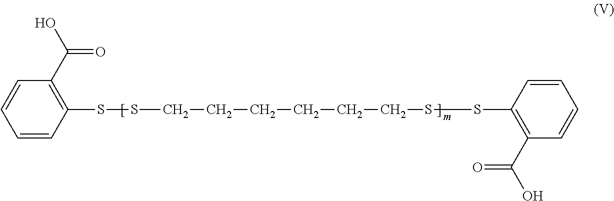

Polysulfides of the invention having the formula (I) in which at least one of the cations K₁⁺ and/or K₂⁺ is/are a cation other than H⁺ can be produced by bringing salts of these cations K₁⁺ and/or K₂⁺ (in particular in the form of their aqueous solutions) into contact with polysulfides of the formula (I). Salts preferably used for this purpose are sulfates, hydrogensulfates, phosphates, hydrogenphosphates, dihydrogenphosphates, carbonates, hydrogencarbonates, hydroxides, nitrates, chlorides, and acetates, particularly sulfates.

Contact with above salts is preferably achieved in an aqueous medium and at temperatures which are preferably from 0 to 20° C., in particular from 0 to 10° C., and preferably at pH values of from 3 to 10, preferably from 4 to 9, in particular from 5 to 8. Polysulfides of the invention having the formula (I) in which K₁⁺ and/or K₂⁺ is/are a cation other than H⁺ can also advantageously be produced in a one-pot process without intermediate isolation of compounds of the formula (V) or their salts.

Polysulfides of the invention having the formula (I) in which K₁⁺ and/or K₂⁺ is/are a cation other than H⁺ are preferably obtained in that, after the compound(s) of the formula (III) has/have been brought into contact with at least one compound of the formula (IV), the polysulfides are precipitated via contact with a salt of the cations K₁⁺ and/or K₂⁺, where K₁⁺ and/or K₂⁺ is/are a cation other than H⁺, preferably with a zinc salt, in particular with zinc sulfate.

The present invention therefore also provides polysulfide compounds of the formula (I) in which K₁⁺ and/or K₂⁺, preferably K₁⁺ and K₂⁺ is/are a cation other than H⁺, preferably being ½Zn²⁺. In particular, the present invention comprises polysulfide compounds of the formula (I) where K₁⁺ and/or K₂⁺ is/are ½Zn²⁺, and m is 1 and/or 2, in particular being 1.

The corresponding polysulfide compounds of the formula (I) where K₁⁺ and/or K₂⁺ is/are a cation other than H⁺, preferably being ½Zn²⁺, are obtainable inter alia via the production process of the invention. Polysulfide compounds of this kind, in particular compounds where m=1, and very particularly preferably the compound where m=1 where K₁⁺ and K₂⁺ are ½Zn²⁺, achieve the effect of the invention when used in rubber mixtures, and they are therefore to be considered as equivalent to the polysulfide mixtures of the invention.

The present process can, in particular in the preferred embodiments, produce the polysulfide mixtures of the invention in very high yield.

It is preferable that the polysulfide mixtures of the invention, based on compounds of the formula (I), are stored at temperatures from 0 to 35° C. after production.

Surprisingly, it has now been found that the polysulfide mixtures of the invention improve the flowability of rubber mixtures and the scorch time thereof, and at the same time give vulcanizates with relatively low rolling resistance and relatively low abrasion.

The invention therefore provides rubber mixtures comprising respectively at least one rubber and one polysulfide mixture of the Invention based on compounds of the formula (I).

In particular, the invention provides rubber mixtures comprising respectively at least one rubber, one sulfur-containing alkoxysilane, one silica-based filler, and one polysulfide mixture of the invention based on compounds of the formula (I). Preferred rubber mixtures comprise the preferred polysulfide mixtures.

The polysulfide mixtures of the Invention can also to some extent or entirely be used after absorption on inert, organic, or inorganic carriers. Preferred carrier materials are silica, natural and synthetic silicates, aluminum oxide, and/or carbon black.

The total content of the polysulfide mixture of the invention in the rubber mixtures of the invention is preferably from 0.1 to 15 phr, particularly from 0.3 to 7 phr, very particularly from 0.5 to 3 phr, and most preferably from 0.7 to 1.5 phr. The unit phr is parts by weight based on 100 parts by weight of rubber used in the rubber mixture.

Natural rubber and/or synthetic rubbers can be used for the production of the rubber mixtures of the invention. Examples of preferred synthetic rubbers are
BR—polybutadiene
ABR—butadlene/C1-C4-alkyl acrylate copolymer
CR—polychioroprene
IR—polyisoprene
SBR—styrene/butadlene copolymers with from 1 to 60% by weight, preferably from 20 to 50% by weight styrene content
IIR—isobutylene/isoprene copolymers
NBR—butadiene/acrylonitrile copolymers having from 5 to 60% by weight, preferably from 10 to 50% by weight, acrylonitrile content
HNBR—partially or fully hydrogenated NBR rubber
EPDM—ethylene/propylene/diene copolymers

and also mixtures of two or more of these rubbers.

It is preferable that the rubber mixtures of the invention comprise at least one SBR and at least one BR, particularly in an SBR:BR ratio by weight of from 60:40 to 90:10.

In one advantageous embodiment, the rubber mixtures of the invention moreover comprise at least one NR. It is particularly preferable that they comprise at least one SBR, at least one BR, and at least one NR, where the ratio by weight of SBR to BR to NR is very particularly preferably from 60 to 85:from 10 to 35:from 5 to 20.

Examples of sulfur-containing alkoxysianes suitable for the rubber mixtures of the invention are bis(triethoxysilylpropyl)tetrasulfane (e.g. Si 69 from Evonik) and bis(triethoxysilylpropyl)disulfane (e.g. Si 75 from Evonik), 3-(triethoxysilyl)-1-propanethiol, polyether-functionalized mercaptosilanes such as Si 363 from Evonik, and thioester-functionalized alkoxysilanes such as NXT or NXT Z from Momentive (previously GE). It is also possible to use mixtures of the sulfur-containing alkoxysilanes. In order to improve ease of metering and/or ease of dispersion, liquid sulfur-containing alkoxysilanes can have been absorbed on a carrier (dry liquid). The content of active ingredient is from 30 to 70 parts by weight, preferably from 40 to 60 parts by weight, for every 100 parts by weight of dry liquid.

The proportion of the sulfur-containing alkoxysilanes in the rubber mixtures of the invention is preferably from 2 to 20 phr, particularly preferably from 3 to 11 phr, and very particularly preferably from 5 to 8 phr, respectively calculated as active ingredient at 100% strength. It is preferable that the quantity of sulfur-containing alkoxysilane is greater than or equal to the quantity of the polysulfide mixture of the invention based on compounds of the formula (I). The ratio by weight of sulfur-containing alkoxysilane to the polysulfide mixture of the invention based on compounds of the formula (I) is particularly preferably from 1.5:1 to 20:1, very particularly preferably from 3:1 to 15:1, and most preferably from 5:1 to 10:1.

The rubber mixture preferred in the invention moreover comprises one or more silica-based fillers. Substances preferably used here are the following:
silica, in particular precipitated silica or fumed silica, produced for example via precipitation of solutions of silicates or flame hydrolysis of silicon halides with specific surface areas of from 5 to 1000 m²/g, preferably from 20 to 400 m²/g (BET surface area) and with primary particle sizes of from 10 to 400 nm. The silicas can optionally also take the form of mixed oxides with other metal oxides such as oxides of Al, of Mg, of Ca, of Ba, of Zn, of Zr, or of Ti.
synthetic silicates such as aluminum silicate, alkaline earth metal silicates such as magnesium silicate or calcium silicate, with BET surface areas of from 20 to 400 m²/g and primary particle size of from 10 to 400 nm,
natural silicates such as kaolin and other naturally occurring silicas,
glass fibers including those in the form of mats and strands,
glass microspheres.

It is of course possible to use additional fillers. Carbon blacks produced by the lamp-black, furnace-black, or gas-black process are particularly suitable for this purpose where the BET surface areas of these are from 20 to 200 m²/g, examples being SAF, ISAF, IISAF, HAF, FEF, or GPF carbon blacks.

The total content of fillers is preferably from 10 to 200 phr, particularly preferably from 50 to 160 phr, and very particularly preferably from 60 to 120 phr.

A particularly preferred embodiment is provided by the combination of silica, carbon black, and polysulfide mixture of the invention. The ratio of silica to carbon black here can vary within any desired limits, but for the application in tires preference is given to a silica:carbon black ratio by weight of from 20:1 to 1.5:1.

In one preferred embodiment, the rubber mixtures of the invention also comprise one or more crosslinking agents. Sulfur-based or peroxidic crosslinking agents are particularly suitable for this purpose, and particular preference is given here to sulfur-based crosslinking agents.

Peroxidic crosslinking agents preferably used are bis(2,4-dichlorobenzyl) peroxide, dibenzoyl peroxide, bis(4-chlorobenzoyl) peroxide, 1,1-bis(tert-butylperoxy)-3,3,5-trimethylcyclohexane, tert-butyl perbenzoate, 2,2-bis(tert-butylperoxy)butane, 4,4-di-tert-butylperoxynonyl valerate, dicumyl peroxide, 2,5-dimethyl-2,5-di(tert-butylperoxy)hexane, tert-butyl cumyl peroxide, 1,3-bis(tert-butylperoxyisopropyl)benzene, di-tert-butyl peroxide, and 2,5-dimethyl-2,5-di(tert-butylperoxy)-3-hexyne.

Other additions that can also be used with advantage, alongside these peroxidic crosslinking agents, are those that can increase crosslinking yield: examples of compounds suitable for this purpose are triallyl isocyanurate, triallyl cyanurate, trimethylolpropane tri(meth)acrylate, triallyl trimellitate, ethylene glycol di(meth)acrylate, butanediol di(meth)acrylate, trimetylolpropane tri(meth)acrylate, zinc diacrylate, zinc dimethacrylate, 1,2-polybutadiene, and N,N′-m-phenylenedimaleimide.

Sulfur can be used as crosslinking agent in elemental soluble or insoluble form, or in the form of sulfur donors. Examples of sulfur donors that can be used are dimorpholyl disulfide (DTDM), 2-morpholinodithlobenzothiazole (MBSS), caprolactam disulfide, dipentamethylenethiuram tetrasulfide (DPTT), and tetramethylthiuram disulfide (TMTD).

The crosslinking of the rubber mixtures of the invention can in principle be achieved with sulfur or sulfur donors alone, or in conjunction with vulcanization accelerators, examples of compounds suitable for these being dithiocarbamates, thiurams, thiazoles, sulfenamides, xanthogenates, bi- or polycyclic amines, guanidine derivatives, dithiophosphates, caprolactams, and thiourea derivatives. Other compounds suitable are moreover zinc diamine diisocyanate, hexamethylenetetramine, 1,3-bis(citraconimidomethyl)benzene, and also cyclic disulfanes. It is preferable that the rubber mixtures of the invention comprise sulfur-based crosslinking agents and vulcanization accelerators.

Crosslinking agents particularly preferably used are sulfur, magnesium oxide, and/or zinc oxide, and the known vulcanization accelerators such as mercaptobenzothiazoles, thiazolsulfenamides, thiurams, thiocarbamates, guanidines, xanthogenates, and thiophosphates are added to these.

Preferred quantities used of the crosslinking agents and vulcanization accelerators are from 0.1 to 10 phr, particularly from 0.1 to 5 phr.

The rubber mixtures of the Invention can comprise other rubber auxiliaries, such as reaction accelerators, aging inhibitors, heat stabilizers, light stabilizers, antioxidants, and in particular antiozonants, flame retardants, processing aids, impact-resistance improvers, plasticizers, tackifiers, blowing agents, dyes, pigments, waxes, extenders, organic acids, retarders, metal oxides, and activators, in particular triethanolamine, polyethylene glycol, hexanetriol, and anti-reversion agents.

The quantities used of these rubber auxiliaries are conventional, depending inter alia on the intended purpose of the vulcanizates. Conventional quantities are from 0.1 to 30 phr.

Preferred aging inhibitors used are alkylated phenols, styrenated phenol, sterically hindered phenols such as 2,6-di-tert-butylphenol, 2,6-di-tert-butyl-p-cresol (BHT), 2,6-di-tert-butyl-4-ethylphenol, sterically hindered phenols containing ester groups, sterically hindered phenols containing thioether, 2,2′-methylenebis-(4-methyl-6-tert-butylphenol) (BPH), and also sterically hindered thiobisphenols.

If discoloration of the rubber is not important, it is also possible to use aminic aging inhibitors, e.g. mixtures of diaryl-p-phenylenediamines (DTPD), octylated diphenylamine (ODPA), phenyl-α-naphthylamine (PAN), phenyl-β-naphthylamine (PBN), preferably those based on phenylenediamine, e.g. N-isopropyl-N′-phenyl-p-phenylenediamine, N-1,3-dimethylbutyl-N′-phenyl-p-phenylenediamine (6PPD), N-1,4-dimethylpentyl-N′-phenyl-p-phenylenediamine (7PPD), N,N′-bis(1,4-dimethylpentyl)-p-phenylenediamine (77PD).

Other aging inhibitors are phosphites such as tris(nonylphenyl)phosphite, polymerized 2,2,4-trimethyl-1,2-dihydroquinoline (TMQ), 2-mercaptobenzimidazole (MBI), methyl-2-mercaptobenzimidazole (MMBI), zinc methylmercaptobenzimidazole (ZMMBI), these mostly being used in combination with above phenolic aging inhibitors. TMQ, MBI, and MMBI are mainly used for NBRs which are vulcanized peroxidically.

Ozone resistance can be improved via antioxidants such as N-1,3-dimethylbutyl-N′-phenyl-p-phenylenediamine (6PPD), N-1,4-dimethylpentyl-N′-phenyl-p-phenylenediamine (7PPD), N,N′-bis(1,4-dimethylpentyl)-p-phenylenediamine (77PD), enol ethers, or cyclic acetals.

Processing aids are intended to act between the rubber particles, and to counteract frictional forces during mixing, plastification, and deformation. The rubber mixtures of the invention can comprise, as processing aids, any of the lubricants that are conventional for the processing of plastics, for example hydrocarbons such as oils, paraffins, and PE waxes, fatty alcohols having from 6 to 20 C atoms, ketones, carboxylic acids, such as fatty acids and montanic acids, oxidized PE wax, metal salts of carboxylic acids, carboxamides, and also carboxylic esters, for example with the alcohols ethanol, fatty alcohols, glycerol, ethanediol, pentaerythritol, and with long-chain carboxylic acids as acid component.

The rubber mixture composition of the invention can also comprise flame retardants in order to reduce flammability and to reduce smoke generation during combustion. Examples of materials used for this purpose are antimony trioxide, phosphoric esters, chloroparaffin, aluminum hydroxide, boron compounds, zinc compounds, molybdenum trioxide, ferrocene, calcium carbonate, and magnesium carbonate.

Prior to crosslinking, it is also possible to add other plastics to the rubber vulcanizate, where these act by way of example as polymeric processing aids or as impact-resistance improvers. These plastics are preferably selected from the group consisting of the homo- and copolymers based on ethylene, propylene, butadiene, styrene, vinyl acetate, vinyl chloride, glycidyl acrylate, glycidyl methacrylate, and on acrylates and methacrylates with alcohol components of branched or unbranched C₁- to C₁₀-alcohols, where particular preference is given to polyacrylates having identical or different alcohol moieties from the group of the C₄- to C₈-alcohol, in particular of butanol, of hexanol, of octanol, and of 2-ethylhexanol, to polymethyl methacrylate, to methyl methacrylate-butyl acrylate copolymers, to methyl methacrylate-butyl methacrylate copolymers, to ethylene-vinyl acetate copolymers, to chlorinated polyethylene, to ethylene-propylene copolymers, and to ethylene-propylene diene copolymers.

In one preferred embodiment, the rubber mixture of the invention comprises from 0.1 to 15 phr of the anti-reversion agent 1,6-bis(N,N-dibenzylthiocarbamoyldithio)hexane (CAS No.: 151900-44-6), with the resultant reduction of tan δ (60° C.), i.e. of rolling resistance, improvement of abrasion values, and reduction of scorch time and of full vulcanization time.

A preferred feature of the rubber mixtures of the invention is that the loss factor tan δ at 60° C. of a vulcanizate produced therefrom under 170° C./t95 heating conditions is <0.16, particularly <0.12, in particular <0.11, while its Shore A hardness at 23° C. is >66. In combination with this, the rubber mixtures of the invention can also achieve a full vulcanization time of less than 2000 seconds.

The present invention further provides a process for the production of rubber mixtures via mixing of at least one rubber with at least one silica-based filler, and one sulfur-containing alkoxysilane, and at least one polysulfide mixture of the Invention. It is preferable here to use from 10 to 150 phr of filler, particularly from 30 to 120 phr, and very particularly from 50 to 100 phr, from 0.1 to 15 phr of polysulfide mixture of the invention, particularly from 0.3 to 7 phr, very particularly from 0.5 to 3 phr, and most preferably from 0.7 to 1.5 phr, and also from 2 to 20 phr of the sulfur-containing alkoxysilane, particularly from 3 to 11 phr, and very particularly preferably from 5 to 8 phr. In the mixing process it is moreover possible to add the abovementioned additional fillers, crosslinking agents, vulcanization accelerators, and rubber auxiliaries, preferably in the quantities stated above.

In the multistage mixing process, the addition of the polysulfide mixture of the invention preferably takes place in the first part of the mixing process, the addition of one or more crosslinking agents, in particular sulfur, and optionally vulcanization accelerators, taking place in a subsequent mixing stage. The temperature of the rubber composition here is preferably from 100 to 200° C., particularly preferably from 120° C. to 170° C. The shear rates for the mixture during mixing are from 1 to 1000 sec⁻¹, preferably from 1 to 100 sec⁻¹. In one preferred embodiment, the rubber mixture is cooled after the first mixing stage, and the crosslinking agent and optionally crosslinking accelerator, and/or additions used to increase crosslinking yield are added in a subsequent mixing stage at <140° C., preferably <100° C. It is equally possible to add the polysulfide mixture of the invention in a subsequent mixing stage and at relatively low temperatures, for example from 40 to 100° C., for example together with sulfur and crosslinking accelerator.

Conventional mixing assemblies, such as rolls, internal mixers, and mixing extruders, can be used to blend the rubber with the filler and with the polysulfide mixture of the invention.

The optional addition of 1,6-bis(N,N-dibenzylthiocarbamoyldithio)hexane preferably takes place in the first stage of the multistage mixing process.

The present invention further provides a process for the vulcanization of the rubber mixtures of the invention which are preferably carried out when temperatures of the composition are from 100 to 200° C., particularly from 130 to 180° C. In one preferred embodiment, the vulcanization takes place at a pressure of from 10 to 200 bar.

The present invention also comprises rubber vulcanizates obtainable via vulcanization of the rubber mixtures of the invention, and also comprises rubber products comprising these vulcanizates, in particular tires, since corresponding tires have the advantage of high hardness coupled with good rolling resistance and with low abrasion.

When vehicles, in particular motor vehicles, are equipped with tires which comprise the vulcanizates of the invention, this leads to lower energy consumption during the operation of these vehicles, thus permitting lower fuel consumption in the case of motor vehicles with internal combustion engines and greater range in the case of vehicles with electric drive, and less effort and/or higher velocity in the case of vehicles driven by muscle power. The present invention therefore also comprises vehicles comprising rubber products which include the vulcanizates of the invention.

The rubber vulcanizates of the invention are suitable for the production of moldings with improved properties, e.g. for the production of cable sheathing, of fuses, of drive belts, of conveyor belts, of roll coverings, of tires, of shoe soles, of sealing means, and of damping elements.

The rubber vulcanizate of the invention can moreover be used for the production of foams. For this, chemical or physical blowing agents are added thereto. Any of the substances known for this purpose can be used as chemical blowing agents, for example azodicarbonamide, p toluenesulfonyl hydrazide, 4,4′-oxybis(benzenesulfohydrazide), p-toluenesulfonyl semicarbazide, 5-phenyltetrazole, N,N′-dinitrosopentamethylenetetramine, zinc carbonate, or sodium hydrogencarbonate, and also mixtures comprising these substances. Examples of physical blowing agents are carbon dioxide and halogenated hydrocarbons.

The present invention further provides the use of the polysulfide mixture of the Invention for the production of rubber mixtures, and of vulcanizates thereof, and in particular for the production of rubber mixtures comprising at least respectively one rubber, one sulfur-containing alkoxysilane, and one silica-based filler.

Surprisingly, it has been found that additive compositions for rubbers comprising at least one sulfur-containing alkoxysilane, in particular bis(triethoxysilylpropyl)tetrasulfane, bis(triethoxysilylpropyl)disulfane, 3-(triethoxysilyl)-1-propanethiol, polyether-functionalized mercaptosilane, or thioester-functionalized alkoxysilane, and the polysulfide mixture of the Invention have sufficient compatibility of the components, despite the reactive groups, thus permitting homogeneous incorporation into rubber mixtures and precise metering in the desired ratio. The present invention therefore also comprises additive compositions of this type for rubbers, and also the use of the polysulfide mixtures of the invention for the production of additive compositions of this type. The ratio by weight of alkoxysilane, in particular of bis(triethoxysilylpropyl)tetrasulfane and/or of bis(triethoxysilylpropyl)disulfane, to the polysulfide mixtures of the invention in these additive compositions is preferably from 1.5:1 to 20:1, particularly preferably from 3:1 to 15:1, and very particularly preferably from 5:1 to 10:1.

The present invention moreover comprises a process for the production of additive compositions for rubbers, characterized in that sulfur-containing alkoxysilanes are mixed with the polysulfide mixtures of the invention.

The present invention further comprises a process for the reduction of the rolling resistance of tires, where a polysulfide mixture of the invention is mixed with a non-crosslinked or partially crosslinked rubber mixture serving as starting material for at least parts of the tire, and the mixture is then vulcanized.

Determination of the Properties of Rubber Mixture and of Vulcanizates:

Measurement of Mooney Viscosity:

The viscosity can be determined directly from the force with which the rubbers (and rubber mixtures) resist processing thereof. In the Mooney shearing disc viscometer, a fluted disc is enclosed, above and below, by test substance and is rotated at about two revolutions per minute in a heatable chamber. The force required here is measured in the form of torque, and corresponds to the respective viscosity. The sample is generally preheated for one minute to 100° C.; the measurement takes a further 4 minutes, the temperature being kept constant here. The viscosity is stated together with the respective test conditions, an example being ML (1+4) 100° C. (Mooney viscosity, large rotor, preheat time and test time in minutes, test temperature).

Scorch Performance (Scorch Time t 5):

The same test can moreover be used as described above to measure the scorch behavior of a mixture. The selected temperature was 130° C. The rotor runs until, after the torque value has passed through a minimum it has increased to 5 Mooney units above the minimum value (t5). The greater the value (unit being seconds), the slower the scorch. An advantageous scorch time in practice is mostly more than 300 seconds.

170° C./t95 Full Vulcanization Time from Rheometer (Vulcameter):

The MDR (moving die rheometer) vulcanization profile and analytical data associated therewith are measured in a MDR 2000 Monsanto rheometer in accordance with ASTM D5289-95. The full vulcanization time determined is the time at which 95% of the rubber has been crosslinked. The selected temperature was 170° C.

Hardness Determination:

The hardness of the rubber mixture of the invention was determined by producing milled sheets of thickness 6 mm from the rubber mixture in accordance with formulations of Table 1.

Test samples of diameter 35 mm were cut from the milled sheets, and the Shore A hardness of these was determined by using a digital Shore hardness tester (Zwick GmbH & Co. KG, Ulm). The hardness of a rubber vulcanizate provides a first indication of its stiffness.

Tensile Test:

The tensile test serves directly to determine the loading limits of an elastomer, and is carried out in accordance with DIN 53504. The increase in length at break is divided by the initial length to give elongation at break. The force for achievement of particular stages of elongation, mostly 50, 100, 200, and 300%, is also determined, and expressed as modulus (tensile strength at the stated elongation of 300%, or 300 modulus).

Dyn. Damping:

Dynamic test methods are used to characterize the deformation behavior of elastomers under periodically changing loads. An externally applied stress changes the conformation of the polymer chain. The loss factor tan δ is determined indirectly here by way of the ratio of loss modulus G″ to storage modulus G′. The loss factor tan δ at 60° C. is associated with rolling resistance and should be as low as possible.

Abrasion:

Abrasion gives an indication of wear, and thus of product lifetime. Abrasion was determined in accordance with DIN 53516. A low value is desirable for economic and environmental reasons.

### EXAMPLE 1

Apparatus: 2000 ml four-necked flask with thermometer, dropping funnel with pressure equalization, reflux condenser with gas-outlet attachment (bubble counter), and tubing, stirrer, gas-inlet tube
Initial charge: 99.1 g (0.25 mol) of Duralink HTS disodium hexamethylene 1,6-bisthiosulfate dihydrate from Flexsys (98.48%)
   600 ml of demineralized water
   41.1 g (0.5 mol) of formaldehyde solution, about 36.5%
   42 g (0.5 mol) of sodium hydrogencarbonate
Feed: 78.7 g (0.5 mol) of 2-mercaptobenzoic acid (98%), dissolved under nitrogen in 500 ml of water at pH 8 via addition of NaOH
Auxiliary: 37% HCl

Duralink HTS and water were used as initial charge in the nitrogen-flushed apparatus. First sodium hydrogencarbonate and then formaldehyde were added, with stirring. The 2-mercaptobenzoic acid solution was then added dropwise at a temperature of from 20 to 25° C. with nitrogen blanketing within about 1 h. After metering had ended, stirring was continued for 22 h, and then pH was adjusted to 2, with nitrogen blanketing at from 20 to 25° C., with 37% hydrochloric acid. The mixture exhibited a very high level of foaming during the pH adjustment. Stirring was continued for one hour, and the solid was then isolated by suction filtration, by using a D4 frit. The product was then washed with portions of in each case 300 ml of water, until the conductivity of the wash water was <0.3 mS/cm, and was then dried at 25° C. in a vacuum drying oven.
Yield: 113.3 g (99.7%) of a mixture of polysulfides of the formula (I) where m=0, 1, and 2

Elemental analysis:

| | | | | |
|---|---|---|---|---|
| C: 52.3% | H: 5.2% | O: 14.3% | S: 28.0% | Cl: 1200 ppm |

The product was analyzed via RP-HPLC and Time-of-Flight Mass Spectrometry (TOF MS). The percentage data relating to the compounds of the formula (I) resulted from the area percentage proportions from the HPLC measurement, using UV detector. The following compounds of the formula (I) were obtained, where K₁⁺ and K₂⁺═H⁺:

25% of compound where m=0; 54% of compound where m=1; 21% of compound where m=2.

### EXAMPLE 2

Apparatus: 4000 ml four-necked flask with thermometer, dropping funnel with pressure equalization, reflux condenser with gas-outlet attachment (bubble counter), and tubing, stirrer, gas-inlet tube, Dulcometer
Initial charge: 198.0 g (0.5 mol) of Duralink HTS disodium hexamethylene 1,6-bisthiosulfate dihydrate from Flexsys (98.59%)
1200 ml of demineralized water
82.3 g (1.0 mol) of formaldehyde solution, about 36.5%
Feed: 157.4 g (1.0 mol) of 2-mercaptobenzoic acid (98%), dissolved under nitrogen in 1000 ml of water via addition of 60 g of NaOH
Auxiliaries: 292 g (0.4 mol) of 5% HCl (metering by way of Dulcometer) 108.4 g (1.1 mol) of 37% HCl

Duralink HTS and water were used as initial charge in the nitrogen-flushed apparatus. Formaldehyde was added with stirring. 2-Mercaptobenzoic acid solution was then added dropwise at a temperature of 5° C. with nitrogen blanketing within about 90 min. The pH of the reaction mixture was kept at from 9.5 to 10.5 during the addition of the 2-mercaptobenzoic acid solution via dropwise addition of 5% HCl. After addition had ended, stirring was continued at 5° C. for 1 h, and then the 37% hydrochloric acid was added dropwise within 1 h with nitrogen blanketing at 5° C. Stirring was continued for one hour, and the solid was then isolated by suction filtration, by using a D4 frit. The product was then washed with portions of in each case 600 ml of water, until the conductivity of the wash water was <0.3 mS/cm, and was then dried at 50° C. in a vacuum drying oven.

Yield: 233 g (102.5%) of a mixture of polysulfides of the formula (I) where m=0, 1, and 2, where K₁⁺ and K₂═H⁺:

Elemental analysis:

| | | | | |
|---|---|---|---|---|
| C: 52.4% | H: 5.1% | O: 14.3% | S: 27.7% | Cl: 130 ppm |

The percentage data relating to the compounds of the formula (I) resulted from the area percentage proportions from the HPLC measurement, using UV detector.

3% of compound where m=0; 96% of compound where m=1; 1% of compound where m=2.

### EXAMPLE 3

Apparatus: 2000 ml four-necked flask with thermometer, dropping funnel with pressure equalization, reflux condenser with gas-outlet attachment (bubble counter), and tubing, stirrer, gas-inlet tube, Dulcometer
Initial charge: 99.0 g (0.25 mol) of Duralink HTS from Flexsys (98.59%)
   600 ml of demineralized water
   41.1 g (0.5 mol) of formaldehyde solution, about 36.5%
Feed: 78.7 g (0.5 mol) of 2-mercaptobenzoic acid (98%), dissolved under nitrogen in 500 ml of water via addition of 30 g of NaOH
Auxiliary: 98.1 g (0.1 mol) of 10% H2SO₄ (metering by way of Dulcometer)
   67.4 g (0.275 mol) of 40% H₂SO₄

Duralink HTS and water were used as initial charge in the nitrogen-flushed apparatus. Formaldehyde was added with stirring. 2-Mercaptobenzoic acid solution was then added dropwise at a temperature of 5° C. with nitrogen blanketing within about 90 min. The pH of the reaction mixture was kept at from 9.5 to 10.5 during the addition of the 2-mercaptobenzoic acid solution via dropwise addition of 10% sulfuric acid. After addition had ended, stirring was continued at 5° C. for 1 h, and then the 40% sulfuric acid was added dropwise within 1 h with nitrogen blanketing at 5° C. Stirring was continued for one hour, and the solid was then isolated by suction filtration, by using a D4 frit. The product was then washed with portions of in each case 300 ml of water, until the conductivity of the wash water was <0.3 mS/cm, and was then dried at 50° C. in a vacuum drying oven.

Yield: 116.7 g (102.7%) of a mixture of polysulfides of the formula (I) where m=0, 1, and 2, where K₁⁺ and K₂⁺═H⁺:

Elemental analysis:

| | | | | |
|---|---|---|---|---|
| C: 52.5% | H: 5.0% | O: 14.5% | S: 28.2% | Cl: <10 ppm |

The percentage data relating to the compounds of the formula (I) resulted from the area percentage proportions from the HPLC measurement, using UV detector.

3% of compound where m=0; 96% of compound m=1; 1% of compound where m=2.

HPLC equipment: Agilent 1100 series with degasser, binary pump, column oven, variable wavelength detector, and autosampler
Stationary phase: Inertsil ODS-3, particle diameter 3 μm
Column length: 150 mm
Internal column diameter: 2.1 mm
Mobile phase A: 100% of water+25 mmol/L of ammonium acetate
   B: 95% of methanol: 5% of water+25 mmol/L of ammonium acetate

Elution Program

| Time (min) | Eluent A (vol %) | Eluent B (vol %) |
|---|---|---|
| 0 | 80 | 20 |
| 5 | 80 | 20 |
| 30 | 1 | 99 |
| 60 | 1 | 99 |
| 62 | 80 | 20 |

Column temperature: 40° C.

Flow rate: 0.3 mV/min

Elution time: 72 min

Injection volume: 5 d

UV detector wavelength: 225 nm

A sample of about 50 mg of product to be analyzed was weighed into a 50 ml graduated flask and 2 ml of dimethyl sulfoxide were admixed, the mixture was made up to the calibration mark with tetrahydrofuran and homogenized, and then directly subjected to chromatography.

### EXAMPLE 4

Apparatus: 2000 ml four-necked flask with thermometer, dropping funnel with pressure equalization, reflux condenser with gas-outlet attachment (bubble counter), and tubing, stirrer, gas-inlet tube, Dulcometer
Initial charge: 99.0 g (0.25 mol) of Duralink HTS from Flexsys (98.59%)
   600 ml of demineralized water
   41.1 g (0.5 mol) of formaldehyde solution, about 36.5%
Feed 1: 78.7 g (0.5 mol) of 2-mercaptobenzoic acid (98%), dissolved under nitrogen in
   500 ml of water via addition of 30 g of NaOH
Auxiliary: 146 g (0.2 mol) of 5% HCl (metering by way of Dulcometer)
Feed 2: 44.8 g (0.25 mol) of ZnSO₄×H₂O in 375 ml of water

Duralink HTS and water were used as initial charge in the nitrogen-flushed apparatus. Formaldehyde was added with stirring. 2-Mercaptobenzoic acid solution was then added dropwise at a temperature of 5° C. with nitrogen blanketing within about 90 min. The pH of the reaction mixture was kept at from 9.5 to 10.5 during the addition of the 2-mercaptobenzoic acid solution via dropwise addition of 5% sulfuric acid. Once addition had ended, stirring was continued at 5° C. for 1 h, and then the zinc sulfate solution was added dropwise within 1 h, with nitrogen blanketing at 5° C. Stirring was continued for one hour, and the solid was then isolated by suction filtration, by using a D4 frit. The product was then washed with portions of in each case 300 ml of water, until the conductivity of the wash water was <0.3 mS/cm, and was then dried at 50° C. in a vacuum drying oven.

Yield: 124.8 g (96.4%) of a mixture of polysulfides of the formula (I) where K₁⁺ and K₂⁺=½Zn²⁺:

Elemental analysis:

| | | | | | |
|---|---|---|---|---|---|
| C: 45.7% | H: 4.2% | O: 13.4% | S: 24.5% | Zn: 12% | Cl: 120 ppm |

The percentage data relating to the compounds of the formula (I) resulted from the area percentage proportions from the HPLC measurement, using UV detector.

4% of compound where m=0; 91% of compound m=1; 5% of compound where m=2.

Production of Rubber Mixtures and of Rubber Vulcanizates

The rubber formulations listed in Table 1 were respectively mixed in accordance with multistage processes described below.

1st Mixing Stage:
BUNA® CB 24 and BUNA® VSL 5025-2 was used as initial charge in an internal mixer and mixed for about 30 seconds
addition of two thirds of VULKASIL® S, two thirds of SI® 69, two thirds of the total quantity of polysulfide mixture of the invention, and mixing for about 60 seconds
addition of one third of VULKASIL® S, one third of SI® 69, one third of the total quantity of polysulfide mixture of the Invention, and also TUDALEN 1849-1, and mixing for about 60 seconds

addition of CORAX® N 339, EDENOR® C 18 98-100, VULKANOX® 4020/LG, VULKANOX® HS/LG, ROTSIEGEL ZINC WHITE, and also ANTILUX® 654, and mixing for about 60 seconds. The mixing temperature was 150° C.

2nd Mixing Stage:

After conclusion of the first mixing stage, the mixture was passed to a downstream roll mill, shaped to give a sheet, and stored for 24 hours at room temperature. The processing temperatures here were below 60° C.

3rd Mixing Stage:

The third mixing stage involved further mastication at 150° C. in a kneader.

4th Mixing Stage:

Addition of the additional substances MAHLSCHWEFEL 90/95 CHANCEL, VULKACIT® CZ/C, VULKACIT® D/C on a roll at temperatures below 80° C.

The rubber mixtures were then fully vulcanized at 170° C. Table 2 gives the properties of the rubber preparations produced and vulcanizates of these.

TABLE 1
Rubber formulation

| | | Rubber | Rubber | Rubber | Rubber |
|---|---|---|---|---|---|
| | Reference | formulation 1 | formulation 2 | formulation 3 | formulation 4 |
| BUNA CB 24 | 30 | 30 | 30 | 30 | 30 |
| BUNA VSL 5025-2 | 96 | 96 | 96 | 96 | 96 |
| CORAX N 339 | 6.4 | 6.4 | 6.4 | 6.4 | 6.4 |
| VULKASIL S | 80 | 80 | 80 | 80 | 80 |
| TUDALEN 1849-1 | 8 | 8 | 8 | 8 | 8 |
| EDENOR C 18 98-100 | 1 | 1 | 1 | 1 | 1 |
| VULKANOX 4020/LG | 1 | 1 | 1 | 1 | 1 |
| VULKANOX HS/LG | 1 | 1 | 1 | 1 | 1 |
| ROTSIEGEL zinc white | 2.5 | 2.5 | 2.5 | 2.5 | 2.5 |
| ANTILUX 654 | 1.5 | 1.5 | 1.5 | 1.5 | 1.5 |
| SI 69 | 6.4 | 6.4 | 6.4 | 6.4 | 6.4 |
| VULKACIT D/C | 2 | 2 | 2 | 2 | 2 |
| VULKACIT CZ/C | 1.5 | 1.5 | 1.5 | 1.5 | 1.5 |
| CHANCEL 90/95 ground | 1.5 | 1.5 | 1.5 | 1.5 | 1.5 |
| sulfur | | | | | |
| Compound of example 1 | | 1 | | | |
| Compound of example 2 | | | 1 | | |
| Compound of example 3 | | | | 1 | |
| Compound of example 4 | | | | | 1 |

| Trade name | Description | Producer/Marketed by |
|---|---|---|
| BUNA CB 24 | BR | Lanxess Deutschland |
| | | GmbH |
| BUNA VSL 5025-2 | SBR | Lanxess Deutschland |
| | | GmbH |
| CORAX N 339 | Carbon black | Degussa-Evonik GmbH |
| VULKASIL S | Silica | Lanxess Deutschland |
| | | GmbH |
| TUDALEN 1849-1 | Mineral oil | Hansen&Rosenthal KG |
| EDENOR C 18 98-100 | Stearic acid | Cognis Deutschland |
| | | GmbH |
| VULKANOX 4020/LG | N-1,3-Dimethylbutyl-N-phenyl-p- | Lanxess Deutschland |
| | phenylenediamine | GmbH |
| VULKANOX HS/LG | Polymerized 2,2,4-trimethyl-1,2- | Lanxess Deutschland |
| | dihydroquinoline | GmbH |
| ROTSIEGEL zinc white | Zinc oxide | Grillo Zinkoxid GmbH |
| ANTILUX 654 | Light stabilizer wax | RheinChemie Rheinau |
| | | GmbH |
| SI 69 | Bis(triethoxysilylpropyl)tetrasulfide | Evonik Industries |
| VULKACIT D/C | 1,3-Diphenylguanidine | Lanxess Deutschland |
| | | GmbH |
| VULKACIT CZ/C | N-Cyclohexyl-2- | Lanxess Deutschland |
| | benzothiazolesulfenamide | GmbH |
| CHANCEL 90/95 ground | Sulfur | Solvay Deutschland |
| sulfur | | GmbH |

Quantitative data in phr (parts by weight per 100 parts of rubber)

TABLE 2
Summary of results

| | | | | Rubber | Rubber | Rubber |
|---|---|---|---|---|---|---|
| | | | | formulation | formulation | formulation |
| Parameter | Unit | DIN | Reference | 1 | 2 | 4 |
| Mooney viscosity | [MU] | 53523 | 95 | 89 | 86 | 87 |
| (ML 1 + 4) | | | | | | |
| Mooney scorch | sec | ASTM | 1253 | 1495 | 1078 | 991 |
| (for 130° C. (t5) | | D5289-95 | | | | |
| Full vulcanization | sec | 53529 | 1417 | 1588 | 1574 | 1523 |
| for 170° C./t95 | | | | | | |
| Shore A | [Shore A] | 53505 | 66 | 72 | 67 | 68 |
| hardness at 23° C. | | | | | | |
| 300 modulus | MPa | 53504 | 15 | 15 | 15 | 15 |
| Elongation at | % | 53504 | 349 | 338 | 322 | 370 |
| break | | | | | | |
| Tensile strength | MPa | 53504 | 19 | 18 | 17 | 20 |
| Abrasion | mm³ | 53516 | 74 | 76 | 72 | 73 |
| Rolling resistance | — | | 0.133 | 0.145 | 0.097 | 0.100 |
| (tan δ (60° C.)) | | | | | | |

In comparison with the reference, the vulcanizates tested exhibit increased hardness values and Improved Mooney viscosity. Rubber formulations 2 and 4 exhibit excellent rolling resistance and improved abrasion.

1. A polysulfide mixture comprising at least one compound of the formula (I), 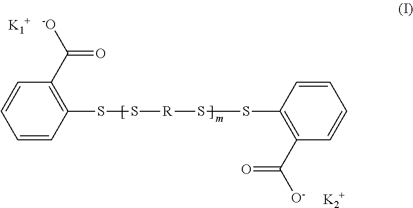
where
R is an alkylene bridge,
K₁⁺ and K₂⁺ are identical or different, and mutually independently are any monovalent cation or the nth part of any n-valent cation, and
M is 1 and optionally 0 and/or 2,
wherein a total proportion of compound(s) where m=1 is at least 80%, based on the total quantity of polysulfide compounds of the formula (I).

2. The polysulfide mixture as claimed in claim 1, wherein:
the mixture comprises two or more compounds of the formula (I):
R is C₆ alkylene; and
K₁⁺ and K₂⁺ are mutually independently H⁺, an alkali metal cation, ½ alkaline earth metal cation, are the nth part of an n-valent rare earth metal cation, or ½Zn²⁺.

3. The polysulfide mixture as claimed in claim 1, wherein the polysulfide mixture may have a chlorine content, and if present, the total chlorine content is <1%.

4. A process for the production of a polysulfide mixture as claimed in claim 1, the process comprising contacting one or more compounds of the formula (III) 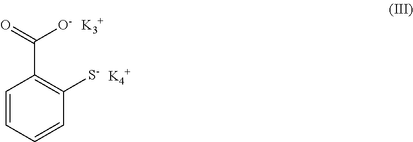
where cations K₃⁺ and K₄⁺ are mutually independently any monovalent cations or are the nth part of any n-valent cation,
with one or more compounds of the formula (IV), 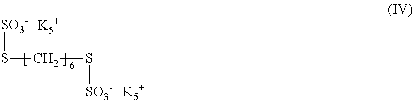
where the two cations K₅⁺ are identical or different, and mutually independently are any monovalent cations or are the nth part of any n-valent cation,
at a temperature of −5° C. to 19° C.

5. The process as claimed in claim 4, wherein for the compound(s) of the formula (III), K₃⁺ and/or K₄⁺≠H⁺, and the process further comprises during the contacting, maintaining a pH at a pH of 7 to 13.

6. The process as claimed in claim 4, further comprising, after contact of the compound of the formula (III) with at least one compound of the formula (IV), precipitating the polysulfide mixture via addition of at least one acid and/or contact with at least one salt of the cations K₁⁺ and/or K₂⁺, where K₁⁺ and/or K₂⁺ is/are a cation other than H⁺.

7. The process as claimed in claim 4, further comprising, after contact of the compound of the formula (III) with at least one compound of the formula (IV), precipitating the polysulfide mixture via contact with at least one zinc salt.

8. A polysulfide compound of the formula (I) as claimed in claim 1, where:
R is C₆ alkylene;
K₁⁺ and K₂⁺ are identical or different, and mutually independently are any monovalent cation or are the nth part of any n-valent cation,
where K₁⁺ and/or K₂⁺, is/are a cation other than H⁺, and
m is 1 or 2.

9. A rubber mixture comprising respectively at least
one rubber; and
one polysulfide mixture as claimed in claim 1, or polysulfide compounds as claimed in claim 8.

10. The rubber mixture as claimed in claim 9 also comprising at least
one sulfur-containing alkoxysilane, and
one silica-based filler.

11. The rubber mixture as claimed in claim 9, further comprising at least one crosslinking agent.

12. The rubber mixture as claimed in claim 9, wherein the rubber comprises, at least one SBR and at least one BR, where the ratio by weight of SBR:BR rubber is 60:40 to 90:10.

13. The rubber mixture as claimed in claim 12, further comprising at least one NR, where the ratio by weight of SBR to BR to NR is 60 to 85:10 to 35:5 to 20.

14. A process for the production of rubber mixtures as claimed in claim 9, the process comprising mixing of at least one rubber with one polysulfide mixture as claimed in claim 1 or polysulfide compounds as claimed in claim 8.

15. A process for the production of vulcanizates, the process comprising vulcanizing the rubber mixture as claimed in claim 9 at a temperature of 100 to 200° C.

16. A vulcanizate obtained via vulcanization of the rubber mixtures as claimed in claim 9.

17. A rubber product comprising one or more rubber vulcanizates as claimed in claim 16.

18. A vehicle comprising a rubber product as claimed in claim 17.

19. (canceled)

20. An additive composition comprising at least one sulfur-containing alkoxysilane, and a polysulfide mixture as claimed in claim 1 or polysulfide compounds as claimed in claim 8.

21. A process for producing the additive composition as claimed in claim 20, the process comprising producing the additive composition from polysulfide mixtures as claimed in claim 1 and/or polysulfide compounds as claimed in claim 8.

22. A process for reducing the rolling resistance of tires, the process comprising mixing a polysulfide mixtures as claimed in claim 1 or polysulfide compounds as claimed in claim 8 with a non-crosslinked or partially crosslinked rubber mixture, forming at least parts of a tire from the rubber mixture, and vulcanizing the mixture.

23. The polysulfide mixture as claimed in claim 2, wherein:
K₁⁺ and K₂+ are identical and are H⁺, an alkali metal cation selected from the group consisting of Li⁺, Na⁺, and K⁺, an ½ alkaline earth metal cation, selected from the group consisting of ½Mg²⁺, ½Ca²⁺, and ⅓Al³⁺, the nth past of an n-valent rare earth metal cation, or ½Zn²⁺;
the proportion of compounds of formula (I) wherein m=1 is at least 85%; and
the polysulfide mixture may have a chlorine content, and if present, the total chlorine content is <0.1%.

24. The polysulfide mixture as claimed in claim 2, wherein:
K₁+ and K₂+ are identical and are H⁺, an alkali metal cation selected from the group consisting of Li⁺, Na⁺, and K⁺, an ½ alkaline earth metal cation, selected from the group consisting of ½Mg²⁺, ½Ca²⁺, and ⅓Al³⁺, the nth part of an n-valent rare earth metal cation, or ½Zn²⁺;
the proportion of compounds of formula (I) wherein m=1 is at least 90%; and
the polysulfide mixture may have a chlorine content, and if present, the total chlorine content is <200 ppm.

25. The polysulfide mixture as claimed in claim 2, wherein:
K₁⁺ and K₂⁺ are identical and are H⁺ or Zn²⁺;
the proportion of compounds of formula (I) wherein m=1 is 93 to 99%; and
the polysulfide mixture may have a chlorine content, and if present, the total chlorine content is <10 ppm.

26. The process according to claim 4, wherein:
cations K₃⁺ and K₄⁺ are mutually independently H⁺, Li⁺, Na⁺, K⁺, ½Mg²⁺, ½Ca²⁺, ⅓Al³⁺, the nth part of an n-valent rare earth metal cation, or ½Zn²⁺,
the two cations K₅⁺ are identical, and are Li⁺, Na⁺, K⁺, ½Mg²⁺, ½Ca²⁺, ⅓Al³⁺, are the nth part of an n-valent rare earth metal cation, or are ½Zn²⁺, and
the contacting is effected at a temperature of 0° C. to 15° C.

27. The process according to claim 4, wherein:
cations K₃⁺ and K₄⁺ are mutually independently H⁺, Na⁺, K⁺ or ½Zn²⁺;
the two cations K₅⁺ are Na⁺; and
the contacting is effected at a temperature of 0° C. to 10° C.

28. The process as claimed in claim 5, wherein maintaining the pH comprises maintaining the pH at a pH of 8 to 12.

29. The process as claimed in claim 5, wherein maintaining the pH comprises maintaining the pH at a pH of 9 to 11 via addition of a Bronsted acid.

30. The process as claimed in claim 15, wherein the temperature is 130 to 180° C.

31. The additive composition as claimed in claim 20, wherein the at least one sulfur-containing alkoxysilane is selected from the group consisting of bis(triethoxysilylpropyl)tetrasulfane, bis(triethoxysilylpropyl)disulfane, 3-(triethoxysilyl)-1-propanethiol, polyether-functionalized mercaptosilane, and thioester-functionalized alkoxysilane.
